(19) United States
(12) Patent Application Publication
(10) Pub. No.: US 2022/0257184 A1
(43) Pub. Date: Aug. 18, 2022

(54) SHORT-TERM VARIABILITY SENSING TO ANTICIPATE TACHYARRHYTHMIAS

(71) Applicant: Medtronic, Inc., Minneapolis, MN (US)

(72) Inventors: Alfonso Aranda Hernandez, Maastricht (NL); Richard N. Cornelussen, Maastricht (NL); Lilian Kornet, Eijsden (NL)

(21) Appl. No.: 17/649,615

(22) Filed: Feb. 1, 2022

Related U.S. Application Data
(60) Provisional application No. 63/150,932, filed on Feb. 18, 2021.

(51) Int. Cl. A61B 5/00; A61B 5/363; A61B 5/355; A61B 5/024; A61B 5/29

(52) U.S. Cl. CPC A61B 5/686; A61B 5/363; A61B 5/355; A61B 5/7203; A61B 5/29; A61B 5/4836; A61B 5/02405

(57) ABSTRACT

Techniques are described including sensing at least one cardiac signal for a patient during a specified time period; determining a short-term variability (STV) metric for the patient based on the at least one cardiac signal sensed during the specified time period, wherein determining the STV metric comprises at least one of: controlling the determined STV metric based on one or more confounding factors, or correcting the determined STV metric based on the one or more confounding factors, wherein the one or more confounders comprise T-wave morphology; and generating a corresponding notification based on the STV metric to one or more computing devices.

### CROSS-REFERENCE TO RELATED APPLICATION

This application claims the benefit of U.S. Provisional Application No. 63/150,932, filed Feb. 18, 2021, and entitled “Short-Term Variability Sensing to Anticipate Tachyarrhythmias,” which is incorporated by reference herein in its entirety.

### FIELD

This disclosure generally relates to medical devices and, more particularly, to detection of tachyarrhythmias by medical devices.

### BACKGROUND

Ventricular tachyarrhythmias are a therapeutic challenge, owing to their relatively unpredictable and deadly nature. Many patients are treated with an implantable cardioverter-defibrillator (ICD) for either primary or secondary prevention of ventricular tachyarrhythmias, meaning those who are at high risk of versus those who have experienced ventricular tachyarrhythmias or sudden cardiac arrest, respectively. Electrodes coupled to the ICD may be placed within or on the heart, and/or at other locations that facilitate delivery of electrical therapy to the heart. The therapy may include anti-arrhythmia pacing, cardiac resynchronization therapy (CRT), defibrillation shock, and/or other types of electrical therapy.

### SUMMARY

In general, the disclosure describes techniques to detect cardiac activity indicative that a tachyarrhythmia will occur prior to occurrence of the tachyarrhythmia. Such techniques may include one or more assessments associated with aspects of a patient's cardiac function, such as determining a short-term variability (STV) metric, and determining a tachyarrhythmia is anticipated based on the outcome of the assessment(s). As an example, a STV metric may be determined based on at least one cardiac signal (e.g., an electrocardiogram (ECG) signal or an electrogram (EGM) signal) received from one or more sensors (e.g., electrodes) of a medical device. Ongoing monitoring of aspects of the patient's cardiac function associated with a patient's condition (e.g., proneness to tachyarrhythmia) may enable detection of changes in cardiac function before such changes lead to tachyarrhythmia and, in some cases, intervention to suppress, e.g., prevent or reduce the likelihood of occurrence of, a tachyarrhythmia event.

Some example techniques may include controlling or correcting for one or more confounding factors which may affect determination of the STV metric or the determination as to whether to deliver therapy configured to suppress tachyarrhythmia based on the STV metric. These confounding factors may include external factors not associated with tachyarrhythmic risk that may influence the cardiac signals sensed by the medical device and thus influence the resultant STV values. The confounding factors may further include other factors that are related to tachyarrhythmic risk that may influence the cardiac signals sensed by the medical device and thus influence the resultant STV values. The confounding factors not associated with tachyarrhythmic risk may include, for example, noise in the cardiac signal and/or T-wave morphology. The confounding factors associated with tachyarrhythmic risk may include, for example, a patient baseline STV, premature ventricular contractions (PVCs), heart rate, circadian variation, and/or medication effects. For example, an increase in cardiac signal noise or poor T-wave morphology may cause variations in the cardiac signal leading to an increase in the calculated STV metric that is not associated with an increase in tachyarrhythmic risk. As another example, PVCs in the cardiac signal, medication effects, or circadian variation may be associated with tachyarrhythmic risk may affect determination of an arrhythmic risk for the patient or whether to deliver therapy configured to suppress tachyarrhythmia based on the determined STV metric.

In some examples, techniques for controlling or correcting for one or more confounding factors may include determining the STV metric based on at least one cardiac signal sensed during a specified time period of the circadian rhythm of a patient. In some examples, the specified time period may be a time period of about 2 hours before and 1 hour after the patient wakes from sleep. For patients who sleep during nighttime hours and usually wake at about 7:00 AM, for example, the specified time period may be in the early morning, such as between 5:00 AM and 8:00 AM. The start and/or end of the specified time period may be adjusted based on the patient's usual waking time. In other examples, the actual wake time of the patient may be detected, and the start and end of the specified time period of the sensed cardiac signal may be determined from the detected actual wake time. For example, the wake time of the patient may be detected based on an activity level of the patient, a heart rate of the patient, a QT interval of the patient or any other manner of determining a wake time of the patient. In other examples, the onset of sleep time of the patient may be detected, and the start and end of the specified time period of the sensed cardiac signal may be determined based on the detected actual onset of sleep time, such as between 5-8 hours after the onset of sleep. The start and/or end of the specified time period may thus be determined in many different ways, including but not limited to a specified time(s) of day, an activity level of the patient, a heart rate of the patient, variations in a QT interval of the patient, a wake time of the patient, and onset of sleep time of the patient, and/or any other method of measuring the circadian rhythm of the patient.

By determining the STV metric based on at least one cardiac signal sensed during the specified time period, the effect of the one or more confounding factors on the STV metric determination may be less as compared to STV metrics based on cardiac signals sensed at other time periods within a patient's circadian rhythm. For example, certain medications (such as beta-blockers) may have less influence over the STV metric at after several hours of sleep due a decrease in the medication effect over time. In addition, the influence of noise on the sensed cardiac signal may be less because of the relatively lower activity level during the hours of sleep as compared with the hours of wakefulness. Thus, according to one or more techniques of the present disclosure, sensing of the cardiac signal during a specified time period may result in a STV metric that is more likely to be associated with tachyarrhythmic risk rather than suggesting an associated tachyarrhythmic risk where none exists or by masking a true association.

In some examples, the disclosure further describes techniques to determine whether to deliver therapy to the patient based on the determined tachyarrhythmia risk. Such techniques may include determining whether to deliver therapy to the patient based on a STV metric determined based on at least one cardiac signal received from one or more sensors of a medical device. In one example, a medical device (such as an IMD) senses at least one cardiac signal of a patient. The at least one cardiac signal may be sensed during a specified time period. The IMD determines, based on the at least one cardiac signal sensed during the specified time period, a STV metric for the patient. The IMD may further determine, based on the STV metric, an arrhythmic risk for the patient and/or whether the IMD should deliver therapy configured to suppress tachyarrhythmia to the patient. For example, the IMD may determine that the STV metric satisfies one or more therapy delivery thresholds and deliver therapy configured to suppress tachyarrhythmia to the patient. The one or more therapy delivery thresholds may be selected such that satisfaction of a therapy delivery threshold is predictive of a tachyarrhythmia event. The therapy delivered to the patient may be selected to suppress, e.g., prevent or reduce the likelihood of occurrence of, the predicted tachyarrhythmia event. In some examples, the therapy may include any type of medical therapy, including but not limited to electrical therapy, drug therapy, magnetic therapy, and/or any other type of non-electrical therapy configured to suppress tachyarrhythmia.

In some examples, in addition to or alternatively to delivering therapy to the patient, the disclosure further describes techniques to determine whether to output a notification to one or more computing device(s) based on the STV metric or an arrhythmic risk level determined based on the STV metric. For example, the techniques of the present disclosure may include determining that the STV metric satisfies one or more notification thresholds and output a notification to one or more computing device(s).

In one example, the disclosure is directed to a method comprising sensing at least one cardiac signal for a patient during a specified time period; determining a short-term variability (STV) metric for the patient based on the at least one cardiac signal sensed during the specified time period, wherein determining the STV metric comprises at least one of: controlling the determined STV metric based on one or more confounding factors, or correcting the determined STV metric based on the one or more confounding factors, wherein the one or more confounders comprise T-wave morphology; and generating a corresponding notification based on the STV metric to one or more computing devices.

In some examples, the method may further include determining that the STV metric satisfies one or more therapy delivery thresholds; and in response to a determination that the STV metric satisfies one or more therapy delivery thresholds, delivering therapy configured to suppress tachyarrhythmia to the patient.

In another example, the disclosure is directed to a medical device comprising sensing circuitry configured to sense at least one cardiac signal of a patient during a specified time period, the specified time period selected to control at least one of noise in the at least one cardiac signal, a circadian variation in the cardiac signal, or a medication effect on the cardiac signal; and processing circuitry configured to: determine a short-term variability (STV) metric for the patient based on the at least one cardiac signal sensed during the specified time period; control the determined STV metric based on one or more confounding factors, or correct the determined STV metric based on the one or more confounding factors, wherein the one or more confounding factors comprise T-wave morphology; and generate a corresponding notification based on the STV metric to one or more computing devices.

In some examples, the processing circuitry may be further configured to determine that the STV metric satisfies one or more therapy delivery thresholds; and in response to a determination that the STV metric satisfies one or more therapy delivery thresholds, deliver therapy configured to suppress tachyarrhythmia to the patient.

In another example, the disclosure is directed to a computer-readable medium comprising instructions that, when executed by one or more processors, cause the one or more processors to: sense at least one cardiac signal for a patient during a specified time period; determine a short-term variability (STV) metric for the patient based on the at least one cardiac signal sensed during the specified time period; control the determined STV metric based on one or more confounding factors, or correct the determined STV metric based on the one or more confounding factors, wherein the one or more confounders comprise T-wave morphology; and generate a corresponding notification including the STV metric to one or more computing devices.

In some examples, the computer-readable medium may further comprise instructions that, when executed by one or more processors, cause the one or more processors to: determine that the STV metric satisfies one or more therapy delivery thresholds; and in response to a determination that the STV metric satisfies one or more therapy delivery thresholds, deliver therapy configured to suppress tachyarrhythmia to the patient.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating an example system that determines a STV metric for a patient in accordance with one or more techniques of the disclosure.

FIG. 2 is a conceptual diagram illustrating the IMD and leads of the system of FIG. 1 in greater detail.

FIG. 3 is a block diagram of an example implantable medical device that determines a STV metric for a patient in accordance with one or more techniques of the disclosure.

FIG. 4 is a block diagram illustrating an example external device that operates in accordance with one or more techniques of the present disclosure.

FIG. 5 is a flowchart illustrating an example process by which a medical device may determine a STV metric based on at least one cardiac signal sensed during a specified time frame and deliver therapy to a patient based on the STV metric in accordance with one or more techniques of the disclosure.

FIG. 6 is a flowchart illustrating an example process by which a computing device may control or correct for one or more confounding factors based on a patient-specific baseline STV value in accordance with one or more techniques of the disclosure.

FIG. 7 is a flowchart illustrating an example process by which a computing device may control or correct for a PVC confounding factor in accordance with one or more techniques of the disclosure.

FIG. 8 is a flowchart illustrating an example process by which a computing device may control or correct for a poor T-wave morphology confounding factor in accordance with one or more techniques of the disclosure.

FIG. 9 is a flowchart illustrating an example process by which a computing device may control or correct for noise as a confounding factor in accordance with one or more techniques of this disclosure.

### DETAILED DESCRIPTION

In general, the disclosure describes example techniques and systems related to determining a tachyarrhythmic risk of a patient based on a short-term variability (STV) metric associated with cardiac function of the patient. For example, processing circuitry of a medical device comprising one or more sensors configured to sense at least one cardiac signal of a patient (e.g., one or more electrodes, accelerometers, or other sensors), or a system that includes the medical device, may determine a STV metric based on the at least one cardiac signal. As used herein, a STV metric may include any metric of the variability or quasiperiodic variation of a time-series of heart beat parameters or cardiac signals, including, but not limited to, heart rate, RR intervals, QT intervals, PR intervals, atrioventricular intervals, or T-wave amplitudes or other T-wave morphological parameters (e.g., T-wave alternans). The variability metric may be a beat-to-beat variability, e.g., determined based on differences between the parameters for adjacent beats, or may be determined using any other techniques for determining variability or quasiperiodic variation of a time series of values or a signal, such as phase-rectified signal averaging.

Some example techniques may include controlling or correcting for one or more confounding factors which may affect determination of the STV metric. These confounding factors may include external factors not related to tachyarrhythmic risk that may influence the cardiac signals sensed by the medical device and thus influence the resultant STV values. The confounding factors may further include other factors that are related to tachyarrhythmic risk that may influence the cardiac signals sensed by the medical device and thus influence the resultant STV values. The confounding factors not related to tachyarrhythmic risk may include, for example, noise in the cardiac signal and/or T-wave morphology. The confounding factors related to tachyarrhythmic risk may include, for example, a patient baseline, presence of premature ventricular contractions, heart rate, circadian variation, and/or medication effects. For example, an increase in cardiac signal noise or poor T-wave morphology may cause variations in the cardiac signal leading to an increase in the calculated STV metric that is not associated with an increase in tachyarrhythmic risk. As another example, presence of PVCs in the cardiac signal, medication effects, or circadian variation may be related to tachyarrhythmic risk and, in addition, their presence may lead to an increase in the calculated STV metric.

In some examples, techniques for controlling or correcting for one or more confounding factors may include determining the STV metric based on at least one cardiac signal sensed during a specified time period. For example, processing circuitry of a medical device comprising one or more sensors (e.g., one or more electrodes, accelerometers, or other sensors), or a system that includes the medical device, may determine a STV metric based on at least one cardiac signal sensed during a specified time period. In general, the specified time period may include a time period of about 2 hours before and 1 hour after the patient wakes from sleep. The start and/or end of the specified time period may be determined based on a time of day, an activity level of the patient, a heart rate of the patient, variations in a QT interval of the patient, and any other method of measuring the circadian rhythm of the patient.

By determining the STV metric based on at least one cardiac signal sensed during the specified time period, the effect of the one or more confounding factors on the STV metric determination may be less as compared to STV metrics based on cardiac signals sensed at other times of day. For example, certain medications (such as beta-blockers) may have less influence over the STV metric at certain times of day due a decrease in medication effect that occurs during the hours of sleep. In addition, the influence of noise on the sensed cardiac signal may be less because of the relatively lower activity level during the hours of sleep as compared with the hours of wakefulness. Thus, according to one or more techniques of the present disclosure, sensing of the cardiac signal during a specified time period may result in an STV metric that is more likely to be associated with tachyarrhythmic risk.

In another example, techniques for controlling or correcting for one or more confounding factors may include determining a patient-specific baseline STV value, selecting one or more patient-specific therapy delivery thresholds based on the patient-specific baseline STV values, determining a STV metric for the patient based on at least one cardiac signal, determining whether the STV metric satisfies one or more of the patient-specific therapy delivery thresholds, and, in response to a determination that the STV metric satisfies one or more of the patient-specific therapy delivery thresholds, delivering therapy configured to suppress tachyarrhythmia to the patient.

In another example, techniques for controlling or correcting for one or more confounding factors may include determining presence of PVCs in the at least one cardiac signal, excluding one or more beats in the cardiac signal where PVCs are present and, in some examples, one or more beats before and after the PVC beats, in order to avoid STV variation due to PVC disturbances, and determining a STV metric based on the at least one cardiac signal in which beats corresponding to presence of PVCs are excluded.

In another example, techniques for controlling or correcting for one or more confounding factors may include determining a T-wave morphology for the at least one cardiac signal, excluding one or more beats in the cardiac signal having poor T-wave morphology, and determining a STV metric based on the at least one cardiac signal in which beats corresponding to poor T-wave morphology are excluded.

In another example, techniques for controlling or correcting for one or more confounding factors may include excluding one or more beats in the cardiac signal in which noise exceeds a threshold, and determining a STV metric based on the at least one cardiac signal in which beats having noise that exceeds a threshold are excluded.

In some examples, the disclosure further describes techniques to determine whether to deliver therapy to the patient based on the determined tachyarrhythmia risk. Such techniques may include determining whether to delivery therapy to the patient based on a STV metric determined based on at least one cardiac signal received from one or more sensors of a medical device. In one example, electrodes associated with a medical device sense at least one cardiac signal of a patient. The at least one cardiac signal may be sensed during a specified time period. Processing circuitry of the medical device comprising one or more sensors (e.g., one or more electrodes, accelerometers, or other sensors), or a system that includes the medical device, may determines a STV metric based on the at least one cardiac signal sensed during the specified time period. The processing circuitry may further determine, based on the STV metric, whether the medical device should deliver therapy configured to suppress tachyarrhythmia to the patient. For example, the processing circuitry may determine that the STV metric satisfies one or more therapy delivery thresholds and cause the medical device to deliver electrical therapy, e.g., cardiac pacing, neurostimulation, or other electrical therapy, to the patient. The one or more therapy delivery thresholds may be selected such that satisfaction of a therapy delivery threshold is predictive of a tachyarrhythmia event. The electrical therapy delivered to the patient may be selected to suppress, e.g., prevent or reduce the likelihood of occurrence of, the predicted tachyarrhythmia event.

In some examples, the medical device may be an implantable medical device (IMD) configured for implantation within the patient. In other examples, the medical device may be an external device. Examples of the one or more implanted or external devices may include an implanted, multi-channel cardiac pacemaker, implantable cardioverter-defibrillator (ICD), implantable pulse generator (IPG), leadless (e.g., intracardiac) pacemaker, extravascular pacemaker and/or ICD, implanted or external neurostimulator, or other IMD or combination of such IMDs, an external monitor, or a drug pump.

Although tachyarrhythmias may be detected during their occurrence and terminated, such techniques may require painful defibrillation shocks and may fail to terminate a potentially deadly tachyarrhythmia. Thus, such other techniques may not enable early detection of changes in such physiological functions and facilitate delivery of therapy configured to suppress tachyarrhythmia before the changes lead to adverse medical events.

Detection of a STV metric determined based on one or more cardiac signals sensed during a specified time period, and that satisfies one or more therapy delivery thresholds, may provide information regarding the tachyarrhythmia risk of the patient not provided by other techniques. For example, a patient's vascular tone may be expected to increase within about 30 minutes after awakening and arising. The increase in vascular tone may reflect higher epinephrine blood levels, which may precipitate adverse medical events. Studies on circadian patterns suggest that changes in vascular tone occurring during the period of about 30 minutes after an increase in patient activity (e.g., after awakening) may reflect changes in the balance between sympathetic activity and vagal tone) and may be identified based on changes in values of a STV metric. Changes in the balance between sympathetic activity and vagal tone may be associated with changes in a tachyarrhythmia risk of the patient. Although monitoring changes in values of a STV metric determined based on at least one cardiac signal sensed during a specified time period thus may enable accurate and/or efficient monitoring of changes in a tachyarrhythmia risk of a patient, such techniques may not readily be carried out during clinician visits.

In some examples, the techniques described herein may enable identification of changes in tachyarrhythmic risk before such changes lead to a tachyarrhythmia. Thus, the techniques described herein may help enable determination of possibility that the patient will experience an adverse medical event, which may lead to delivery of therapy designed to suppress, e.g., prevent or reduce the likelihood of occurrence of, the adverse medical event.

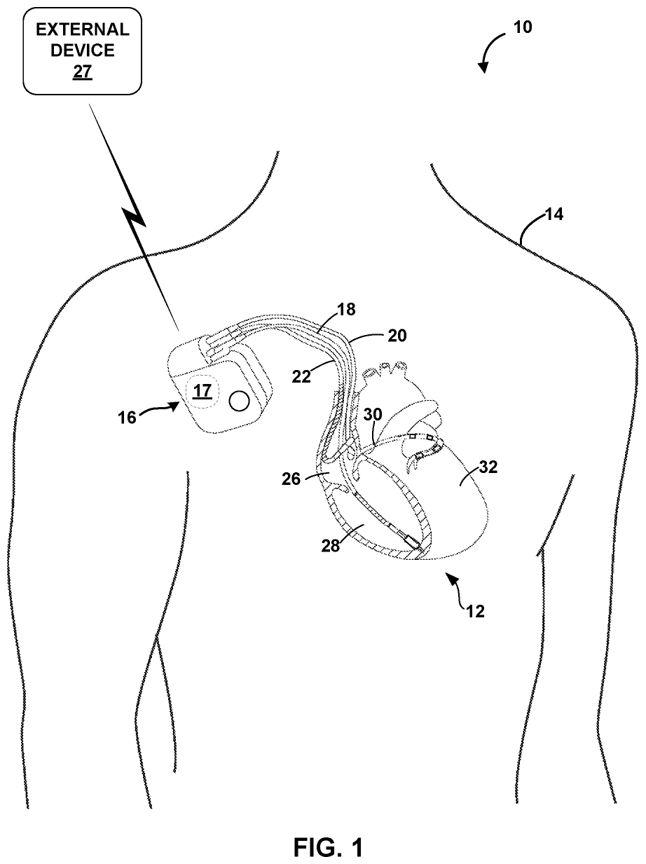

FIG. 1 is a conceptual diagram illustrating an example system that determines a STV metric for a patient in accordance with one or more techniques of the disclosure. Medical device system 10 includes IMD 16 and external device 27. As illustrated by example system 10 in FIG. 1, IMD 16 may, in some examples, be an implantable cardiac pacemaker, implantable cardioverter/defibrillator (ICD), or pacemaker/cardioverter/defibrillator, for example. IMD 16 is connected to leads 18, 20 and 22. IMD 16 is communicatively coupled, e.g., capable of being selectively communicatively coupled, to external device 27. Although not illustrated in FIG. 1, external device 27 may be communicatively coupled to one or more computing devices over a communication network.

IMD 16 senses electrical signals attendant to the depolarization and repolarization of heart 12, e.g., a cardiac electrogram (EGM), via electrodes on one or more leads 18, 20 and 22 or the housing of IMD 16. IMD 16 may also deliver therapy in the form of electrical signals to heart 12 via electrodes located on one or more leads 18, 20 and 22 or a housing of IMD 16. The therapy may be pacing, cardioversion and/or defibrillation pulses. IMD 16 may monitor EGM signals collected by electrodes on leads 18, 20 or 22, and based on the EGM signals, diagnose, and treat cardiac episodes, such as tachyarrhythmias.

In some examples, IMD 16 includes communication circuitry 17 including any suitable circuitry, firmware, software, or any combination thereof for communicating with another device, such as external device 27 of FIG. 1. For example, communication circuitry 17 may include one or more processors, memory, wireless radios, antennae, transmitters, receivers, modulation and demodulation circuitry, filters, amplifiers, or the like for radio frequency communication with other devices, such as external device 27. IMD 16 may use communication circuitry 17 to receive downlinked data from external device 27 to control one or more operations of IMD 16 and/or send uplinked data to external device 27.

Leads 18, 20, 22 extend into the heart 12 of patient 14 to sense electrical activity of heart 12 and/or deliver electrical therapy to heart 12. In the example shown in FIG. 1, right ventricular (RV) lead 18 extends through one or more veins (not shown), the superior vena cava (not shown), and right atrium 26, and into right ventricle 28. Left ventricular (LV) lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12.

While example system 10 of FIG. 1 depicts IMD 16, in other examples, the techniques of the disclosure may be applied to other types of medical devices that are not necessarily implantable. For example, a medical device in accordance with one or more techniques of the disclosure may include a wearable medical device or “smart” apparel worn by patient 14. For example, such a medical device may take the form of a wristwatch worn by patient 14, circuitry that is adhesively affixed to patient 14, or a wearable automated external defibrillator (WAED). In another example, a medical device as described herein may include an external medical device with implantable electrodes.

In some examples, external device 27 takes the form of an external programmer or mobile device, such as a mobile phone, a “smart” phone, a laptop, a tablet computer, a personal digital assistant (PDA), a wearable electronic device, a handheld computing device, computer workstation, server or other networked computing device, etc. In some examples, external device 27 is a CareLink™ monitor available from Medtronic, Inc. While depicted as a single device in the example of FIG. 1, in some examples, external device 27 comprises one or more computing devices that implement a remote monitoring or remote care system. A user, such as a physician, technician, surgeon, electro-physiologist, or other clinician, may interact with external device 27 to retrieve physiological or diagnostic information from IMD 16. A user, such as patient 14 or a clinician as described above, may also interact with external device 27 to program IMD 16, e.g., select or adjust values for operational parameters of IMD 16. External device 27 may include processing circuitry, a memory, a user interface, and communication circuitry capable of transmitting and receiving information to and from IMD 16.

IMD 16 and external device 27 may communicate via wireless communication using any techniques known in the art. Examples of communication techniques may include, for example, include radiofrequency (RF) telemetry, which may be an RF link established via an antenna according to Bluetooth® or Bluetooth® Low Energy (BLE)®, WiFi, or medical implant communication service (MICS), though other techniques are also contemplated. In some examples, external device 27 may include a programming head that may be placed proximate to the patient's body near the IMD 16 implant site in order to improve the quality or security of communication between IMD 16 and external device 27.

In accordance with one or more techniques of the disclosure, medical device system 10 (e.g., IMD 16, external device 27, or both) determines an STV metric of the patient based on at least one cardiac signal. In some examples, to control or correct for one or more confounding factors in the STV determination, the at least one cardiac signal may be sensed during a specified time period. Medical device system 10 may further determine whether the STV metric satisfies one or more therapy delivery thresholds, and, in response to a determination that the STV metric satisfies one or more therapy delivery thresholds, deliver therapy configured to suppress tachyarrhythmia to patient 14. In some examples, the at least one cardiac signal sensed by medical device 10 includes a cardiac electrogram signal, such as an electrocardiogram (ECG) signal, of patient 14. The one or more therapy delivery thresholds may be selected such that satisfaction of a therapy delivery threshold is predictive of a tachyarrhythmia event. The therapy delivered to the patient may be selected to help suppress, e.g., prevent or reduce the likelihood of occurrence of, the predicted tachyarrhythmia event.

Medical device system 10 is described as including IMD 16 and external device 27. External device 27 may include any type of computing device such as a mobile phone, a “smart” phone, a laptop, a tablet computer, a personal digital assistant (PDA), a wearable electronic device, a handheld computing device, computer workstation, an external programmer, external therapy or monitoring device, or any other type of computing device.

In another example, medical device system 10 may control or correct for one or more confounding factors may by determining a patient-specific baseline STV value, selecting one or more patient-specific therapy delivery thresholds based on the patient-specific baseline STV values, determining a STV metric for the patient based on at least one cardiac signal, determining whether the STV metric satisfies one or more of the patient-specific therapy delivery thresholds, and, in response to a determination that the STV metric satisfies one or more of the patient-specific therapy delivery thresholds, delivering therapy configured to suppress tachyarrhythmia to the patient.

In another example, medical device system 10 may control or correct for one or more confounding factors may by determining presence of PVCs in the at least one cardiac signal, excluding one or more beats in the cardiac signal where PVCs are present, and determining a STV metric based on the at least one cardiac signal in which beats corresponding to presence of PVCs are excluded.

In another example, medical device system 10 may control or correcting for one or more confounding factors by determining a T-wave morphology for the at least one cardiac signal, excluding one or more beats in the cardiac signal having poor T-wave morphology, and determining a STV metric based on the at least one cardiac signal in which beats corresponding to poor T-wave morphology are excluded.

By determining the STV metric using any one or more of the confounder control or correction techniques of this disclosure, the effect of the one or more confounding factors on the STV metric determination may be less as compared to STV metrics that are not determined using the confounder control or correction techniques described herein. Thus, according to one or more techniques of the present disclosure, determination of a STV metric using any one or more of the techniques of the present disclosure may result in an STV metric that is more likely to be associated with tachyarrhythmic risk. In turn, the determination as to whether electrical therapy based on the STV metric is more likely to result in a determination that therapy should be delivered when the tachyarrhythmic risk is actually increased. In this way, the techniques of the disclosure may help to prevent false positives in the determination of whether a patient is at risk of tachyarrhythmia or more serious cardiac event, further helping to prevent delivery of unnecessary electrical therapy and avoiding unnecessary physical and psychological distress for the patient.

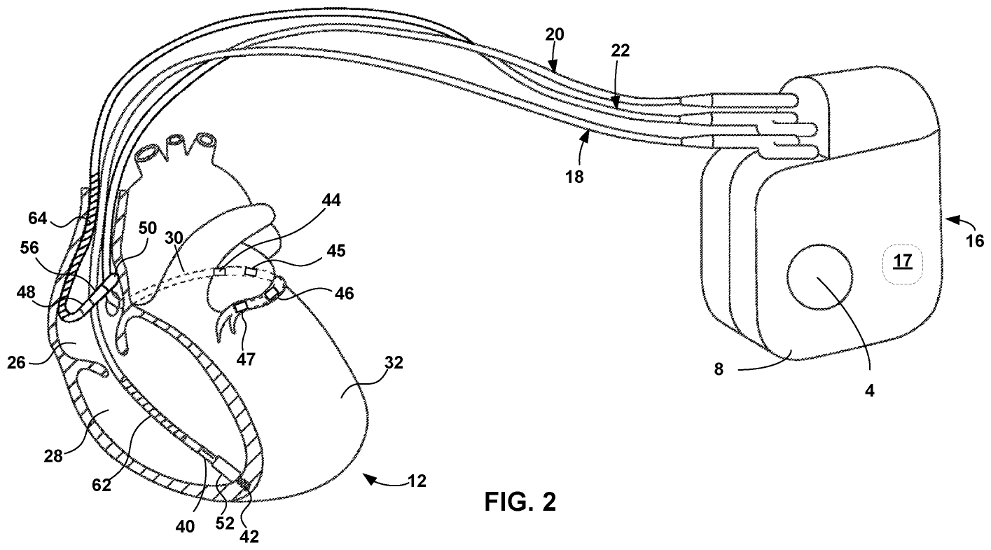

FIG. 2 is a conceptual diagram illustrating IMD 16 and leads 18, 20, 22 of system 10 of FIG. 1 in greater detail. In the illustrated example, bipolar electrodes 40 and 42 are located adjacent to a distal end of lead 18, and bipolar electrodes 48 and 50 are located adjacent to a distal end of lead 22. In addition, four electrodes 44, 45, 46 and 47 are located adjacent to a distal end of lead 20. Lead 20 may be referred to as a quadrapolar LV lead. In other examples, lead 20 may include more or fewer electrodes. In some examples, LV lead 20 comprises segmented electrodes, e.g., in which each of a plurality of longitudinal electrode positions of the lead, such as the positions of electrodes 44, 45, 46 and 47, includes a plurality of discrete electrodes arranged at respective circumferential positions around the circumference of lead.

In the illustrated example, electrodes 40 and 44-48 take the form of ring electrodes, and electrodes 42 and 50 may take the form of extendable helix tip electrodes mounted retractably within insulative electrode heads 52 and 56, respectively. In some examples, each of electrodes 40, 42, 44-48, and 50 is electrically coupled to a respective conductor within the lead body of its associated lead 18, 20, 22 and thereby coupled to circuitry within IMD 16.

In some examples, IMD 16 includes one or more housing electrodes, such as housing electrode 4 illustrated in FIG. 2, which may be formed integrally with an outer surface of hermetically-sealed housing 8 of IMD 16 or otherwise coupled to housing 8. In some examples, housing electrode 4 is defined by an uninsulated portion of an outward facing portion of housing 8 of IMD 16. Other divisions between insulated and uninsulated portions of housing 8 may be employed to define two or more housing electrodes. In some examples, a housing electrode comprises substantially all of housing 8.

Housing 8 encloses signal generation circuitry that generates therapeutic signals, such as cardiac pacing, cardioversion, and defibrillation pulses, as well as sensing circuitry for sensing electrical signals attendant to the depolarization and repolarization of heart 12. Housing 8 may also enclose a memory for storing the sensed electrical signals. Housing 8 may also enclose a communication circuitry 17 for communication between IMD 16 and external device 27 (see FIG. 1).

IMD 16 senses electrical signals attendant to the depolarization and repolarization of heart 12 via electrodes 4, 40, 42, 44-48, and 50. IMD 16 may sense such electrical signals via any bipolar combination of electrodes 40, 42, 44-48, and 50. Furthermore, any of the electrodes 40, 42, 44-48, and 50 may be used for unipolar sensing in combination with housing electrode 4.

The illustrated numbers and configurations of leads 18, 20 and 22 and electrodes are merely examples. Other configurations, i.e., number and position of leads and electrodes, are possible. In some examples, system 10 may include an additional lead or lead segment having one or more electrodes positioned at different locations in the cardiovascular system for sensing and/or delivering therapy to patient 14. For example, instead of or in addition to intracardiac leads 18, 20 and 22, system 10 may include one or more epicardial or extravascular (e.g., subcutaneous or substernal) leads not positioned within heart 12.

In accordance with one or more techniques of the disclosure, IMD 16 (and/or external device 27 of FIG. 1, or both) determines an STV metric of the patient based on at least one cardiac signal. In some examples, to control or correct for one or more confounding factors, the at least one cardiac signal may be sensed during a specified time period. In some examples, the at least one cardiac signal sensed by IMD 16 includes a cardiac electrogram signal, such as an electrocardiogram (ECG) signal, of a patient 14 (see FIG. 1). IMD 16 may further determine whether the STV metric satisfies one or more therapy delivery thresholds, and, in response to a determination that the STV metric satisfies one or more therapy delivery thresholds, deliver therapy configured to suppress tachyarrhythmia to the patient. The one or more therapy delivery thresholds may be selected such that satisfaction of a therapy delivery threshold is predictive of a tachyarrhythmia event. The therapy delivered to the patient may be selected to help suppress, e.g., prevent or reduce the likelihood of occurrence of, the predicted tachyarrhythmia event.

In another example, IMD 16 may control or correct for one or more confounding factors by determining a patient-specific baseline STV value, selecting one or more patient-specific therapy delivery thresholds based on the patient-specific baseline STV values, determining a STV metric for the patient based on at least one cardiac signal, determining whether the STV metric satisfies one or more of the patient-specific therapy delivery thresholds, and, in response to a determination that the STV metric satisfies one or more of the patient-specific therapy delivery thresholds, delivering therapy configured to suppress tachyarrhythmia to the patient.

In another example, IMD 16 may control or correct for one or more confounding factors by determining presence of PVCs in the at least one cardiac signal, excluding one or more beats in the cardiac signal where PVCs are present, and determining a STV metric based on the at least one cardiac signal in which beats corresponding to presence of PVCs are excluded.

In another example, IMD 16 may control or correct for one or more confounding factors by determining a T-wave morphology for the at least one cardiac signal, excluding one or more beats in the cardiac signal having poor T-wave morphology, and determining a STV metric based on the at least one cardiac signal in which beats corresponding to poor T-wave morphology are excluded.

Although described herein in the context of example IMD 16 that provides electrical therapy, the techniques disclosed herein may be used with other types of devices and/or therapy delivering devices. For example, the techniques may be implemented with one or more of an extra-cardiac defibrillator coupled to electrodes outside of the heart or outside of the cardiovascular system, a transcatheter pacemaker configured for implantation within the heart, such as the Micra™ transcatheter pacing system commercially available from Medtronic PLC of Dublin Ireland, an insertable cardiac monitor, such as the Reveal LINQ™ ICM, also commercially available from Medtronic PLC, a neurostimulator, a drug delivery device, a wearable device such as a wearable cardioverter defibrillator, wearable ECG monitor, a fitness tracker, or other wearable and/or “smart” devices, a mobile device, such as a mobile phone, a “smart” phone, a laptop, a tablet computer, a personal digital assistant (PDA), or “smart” apparel such as “smart” glasses or a “smart” watch.

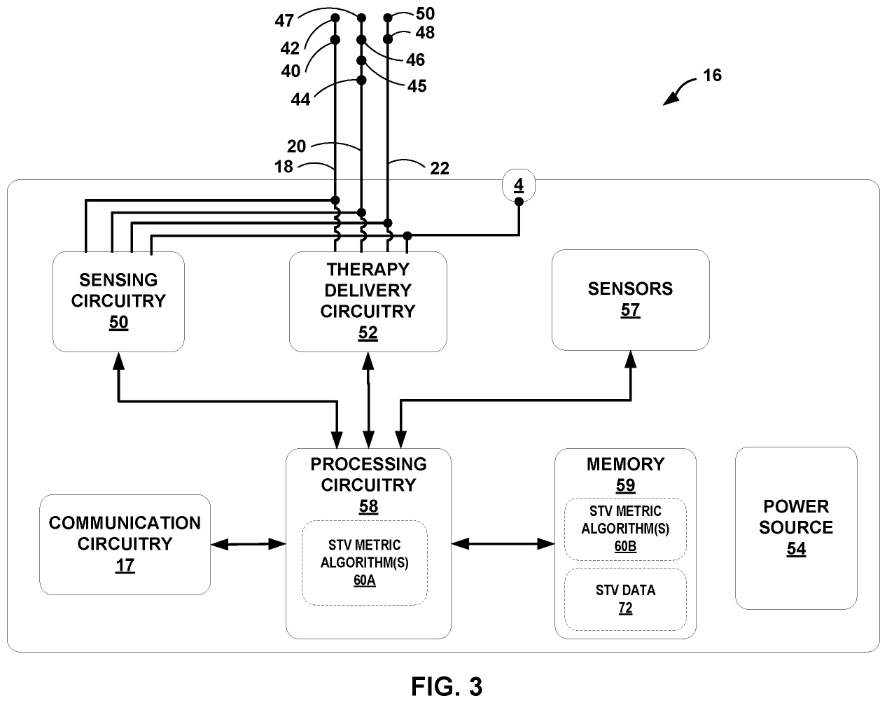

FIG. 3 is a block diagram of an example IMD 16 according to one or more techniques of the disclosure. In the illustrated example, IMD 16 includes processing circuitry 58, memory 59, communication circuitry 17, sensing circuitry 50, therapy delivery circuitry 52, sensors 57, and power source 54. Memory 59 includes computer-readable instructions that, when executed by processing circuitry 58, cause IMD 16 and processing circuitry 58 to perform various functions attributed to IMD 16 and processing circuitry 58 herein. For example, the computer readable instructions may cause IMD 16 and/or processing circuitry 58 to sense at least one cardiac signal during a specified period of time, determine a STV metric based on the at least one cardiac signal sensed during the specified period of time, determine, based on the STV metric, whether to deliver therapy configured to suppress tachyarrhythmia, and deliver the therapy. Memory 59 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processing circuitry 58 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 58 may include multiple components, such as any combination of one or more microprocessors, one or more controllers, one or more DSPs, one or more ASICs, or one or more FPGAs, as well as other discrete or integrated logic circuitry. The functions attributed to processing circuitry 58 herein may be embodied as software, firmware, hardware or any combination thereof.

Processing circuitry 58 controls therapy delivery circuitry 52 to deliver therapy to heart 12 according to therapy parameters, which may be stored in memory 59. For example, processing circuitry 58 may control therapy delivery circuitry 52 to deliver electrical pulses with the amplitudes, pulse widths, frequency, or electrode polarities specified by the therapy parameters. In this manner, therapy delivery circuitry 52 may deliver pacing pulses (e.g., overdrive pacing pulses, ATP pulses, bradycardia pacing pulses, or post-shock pacing therapy) to heart 12 via one or more of electrodes 4, 40, 42, 44-48, and 50. In some examples, therapy delivery circuitry 52 may additionally or alternatively deliver neurostimulation. In some examples, therapy delivery circuitry 52 may deliver such electrical stimulation in the form of voltage or current electrical pulses. In other examples, therapy delivery circuitry 52 may deliver one or more of these types of stimulation in the form of other signals, such as sine waves, square waves, or other substantially continuous time signals.

In some examples, processing circuitry 58 may implement one or more STV metric algorithms 60A stored by memory 59 as STV metric algorithm 60B (collectively, STV metric algorithms 60). Processing circuitry 58 applies a STV metric algorithm 60 to determine a STV metric based on at least one cardiac signal sensed during a specified time frame.

Therapy delivery circuitry 52 is electrically coupled to electrodes 4, 40, 42, 44-48, and 50. In other examples, IMD 16 may utilize other numbers of electrodes not depicted in FIG. 3. IMD 16 may use any combination of electrodes to deliver electrical therapy and/or detect electrical signals from patient 12. In some examples, therapy delivery circuitry 52 includes a charging circuit, one or more pulse generators, capacitors, transformers, switching modules, and/or other components capable of generating and/or storing energy to deliver as pacing therapy, cardiac resynchronization therapy, other therapy or a combination of therapies. In some examples, therapy delivery circuitry 52 delivers therapy as one or more electrical pulses according to one or more therapy parameter sets defining an amplitude, a frequency, a voltage or current of the therapy, or other parameters of the therapy.

Sensing circuitry 50 monitors signals from one or more combinations (also referred to as vectors) of two or more electrodes from among electrodes 4, 40, 42, 44-48, and 50 in order to monitor electrical activity of heart 12, impedance, or other electrical phenomenon. In some examples, sensing circuitry 50 includes one or more analog components, digital components or a combination thereof. In some examples, sensing circuitry 50 includes one or more sense amplifiers, comparators, filters, rectifiers, threshold detectors, analog-to-digital converters (ADCs) or the like. In some examples, sensing circuitry 50 converts sensed signals to digital form and provides the digital signals to processing circuitry 58 for processing or analysis. In one example, sensing circuitry 50 amplifies signals from electrodes 4, 40, 42, 44-48, and 50 and converts the amplified signals to multi-bit digital signals by an ADC.

In some examples, sensing circuitry 50 performs sensing of the cardiac electrogram to determine heart rates, detect arrhythmias (e.g., tachyarrhythmias or bradycardia), determine STV metrics, or to sense other parameters or events from the cardiac electrogram. Sensing circuitry 50 may also include a switching circuitry to select which of the available electrodes (and the electrode polarity) are used to sense the heart activity, depending upon which electrode combination, or electrode vector, is used in the current sensing configuration. Processing circuitry 58 may control the switching circuitry to select the electrodes that function as sense electrodes and their polarity. Sensing circuitry 50 may include one or more detection channels, each of which may be coupled to a selected electrode configuration for detection of cardiac signals via that electrode configuration. In some examples, sensing circuitry 50 compares processed signals to a threshold to detect the existence of atrial or ventricular depolarizations and indicate the existence of the atrial depolarization (e.g., P-waves) or ventricular depolarizations (e.g., R-waves) to processing circuitry 58. Sensing circuitry 50 may comprise one or more amplifiers or other circuitry for comparison of the cardiac electrogram amplitude to a threshold, which may be adjustable.

In some examples, sensors 57 may comprise one or more of an accelerometer, pressure sensor, or optical sensor. In some examples, sensors 57 may provide a signal to processing circuitry 58 indicative of the mechanical activity of the heart. Although described herein with respect to examples in which the cardiac signal is a cardiac EGM or other signal indicative of the electrical activity of the heart, STV metrics may be determined based on a cardiac signal indicative of the mechanical activity of the heart in some examples.

Processing circuitry 58 may include a timing and control module, which may be embodied as hardware, firmware, software, or any combination thereof. The timing and control module may comprise a dedicated hardware circuit, such as an ASIC, separate from other processing circuitry 58 components, such as a microprocessor, or a software module executed by a component of processing circuitry 58, which may be a microprocessor or ASIC. The timing and control module may implement programmable counters. If IMD 16 is configured to generate and deliver bradycardia pacing pulses to heart 12, such counters may control the basic time intervals associated with DDD, VVI, DVI, VDD, AAI, DDI, DDDR, VVIR, DVIR, VDDR, AAIR, DDIR and other modes of pacing.

Memory 59 may be configured to store a variety of operational parameters, therapy parameters, sensed and detected data, and any other information related to the therapy and treatment of patient 12. In the example of FIG. 3, memory 59 may store sensed cardiac EGM signals sensed during one or more time periods. Memory 59 may further store one or more therapy parameters that define the delivery of therapy provided by therapy delivery circuitry 52. In other examples, memory 59 may act as a temporary buffer for storing data until it can be uploaded to external device 27 of FIG. 1.

In accordance with the techniques of the present disclosure, memory 59 may store STV data 72. STV data 72 may include, for example, one or more STV metrics calculated for the patient. STV data 72 may further include one or more therapy delivery thresholds. When processing circuitry 58 determines that a STV metric satisfies one or more of the therapy delivery thresholds, processing circuitry may cause therapy delivery circuitry 52 to deliver electrical therapy configured to suppress tachyarrhythmia to the patient via one or more of electrodes 40, 42, 44-48, and/or 50. The one or more therapy delivery thresholds may be specific to each patient. For example, the one or more therapy delivery thresholds for a patient may be determined based on STV baseline measurements for the patient during one or more periods of time when no tachyarrhythmia events occurred.

A STV metric may be a measure of variability within a set of values of a parameter of a cardiac signal collected during a measurement period, with each value corresponding to a respective one of a plurality of heart beats. In some examples, the parameter may relate to repolarization of the heart during the beat. Example parameters include heart rate or RR interval, QT interval, PR duration, the time between the end of the QRS complex and the end of the T-wave, or T-wave morphology parameters. Example T-wave morphology parameters include T-wave amplitude, T-wave width, the interval between a maximum positive value of the derivative of the T-wave and a maximum negative value of the derivative of the T-wave, an area under the curve measurement of the T-wave, or a ratio of an area under the curve of a positive portion of the derivative of the T-wave to an area under the curve of a negative portion of the derivative of the T-wave. Other example parameters may include a ratio of an area under the curve of a positive portion of the derivative of the EGM or ECG signal to an area under the curve of a negative portion of the derivative of the EGM or ECG signal. Processing circuitry may determine the values in the set based on indications of the occurrence of features in the cardiac EGM, such as R-waves and/or P-waves, from sensing circuitry 50, or based on an analysis of a digitized version of the cardiac EGM received from sensing circuitry 50. Processing circuitry 58 may determine the variability of the values in the set using any known variability calculation techniques.

Communication circuitry 17 includes any suitable circuitry, firmware, software, or any combination thereof for communicating with another device, such as external device 27 of FIG. 1. For example, communication circuitry 17 may include one or more antennae, modulation and demodulation circuitry, filters, amplifiers, or the like for radio frequency communication with other devices, such as external device 27. Under the control of processing circuitry 58, communication circuitry 17 may receive downlink telemetry from and send uplink telemetry to external device 27 with the aid of an antenna, which may be internal and/or external. Processing circuitry 58 may provide the data to be uplinked to external device 27 and the control signals for the telemetry circuit within communication circuitry 17, e.g., via an address/data bus. In some examples, communication circuitry 17 may provide received data to processing circuitry 58 via a multiplexer.

Power source 54 may be any type of device that is configured to hold a charge to operate the circuitry of IMD 16. Power source 54 may be provided as a rechargeable or non-rechargeable battery. In other example, power source 54 may incorporate an energy scavenging system that stores electrical energy from movement of IMD 16 within patient 14.

In accordance with one or more techniques of the disclosure, IMD 16 determines an STV metric of the patient based on at least one cardiac signal sensed during a specified time period. IMD 16 may further determine whether the STV metric satisfies one or more STV therapy delivery thresholds, and, in response to a determination that the STV metric satisfies one or more of the STV therapy delivery thresholds, deliver therapy configured to suppress tachyarrhythmia to patient 14. For example, the one or more STV therapy delivery thresholds may be stored in memory 59. The one or more therapy delivery thresholds may be selected such that satisfaction of a STV metric therapy delivery threshold is predictive of a tachyarrhythmia event. The therapy delivered to the patient may be selected to suppress, e.g., prevent or reduce the likelihood of occurrence of, the predicted tachyarrhythmia event.

By determining the STV metric based on at least one cardiac signal sensed during the specified time period, the effect of the one or more confounding factors on the STV metric determination may be less as compared to STV metrics determined based on cardiac signals sensed at other times of day. Thus, according to one or more techniques of the present disclosure, sensing of the cardiac signal during a specified time period may result in an STV metric that is more likely to be associated with tachyarrhythmic risk. In turn, the determination as to whether to deliver therapy configured to suppress tachyarrhythmia (wherein the determination is based on the STV metric) is more likely to result in delivery of therapy when the tachyarrhythmic risk is actually increased. In this way, one or more techniques of the disclosure may help to prevent false positives in the determination of whether a patient is at risk of tachyarrhythmia or more serious cardiac event, further helping to prevent delivery of unnecessary electrical therapy and avoiding unnecessary physical and psychological distress for the patient.

In another example, IMD 16 may control or correct for one or more confounding factors by determining a patient-specific baseline STV value, selecting one or more patient-specific therapy delivery thresholds based on the patient-specific baseline STV values, determining a STV metric for the patient based on at least one cardiac signal, determining whether the STV metric satisfies one or more of the patient-specific therapy delivery thresholds, and, in response to a determination that the STV metric satisfies one or more of the patient-specific therapy delivery thresholds, delivering therapy configured to suppress tachyarrhythmia to the patient.

In another example, IMD 16 may control or correct for one or more confounding factors by determining presence of PVCs in the at least one cardiac signal, excluding one or more beats in the cardiac signal where PVCs are present, and determining a STV metric based on the at least one cardiac signal in which beats corresponding to presence of PVCs are excluded.

In another example, IMD 16 may control or correct for one or more confounding factors by determining a T-wave morphology for the at least one cardiac signal, excluding one or more beats in the cardiac signal having poor T-wave morphology, and determining a STV metric based on the at least one cardiac signal in which beats corresponding to poor T-wave morphology are excluded.

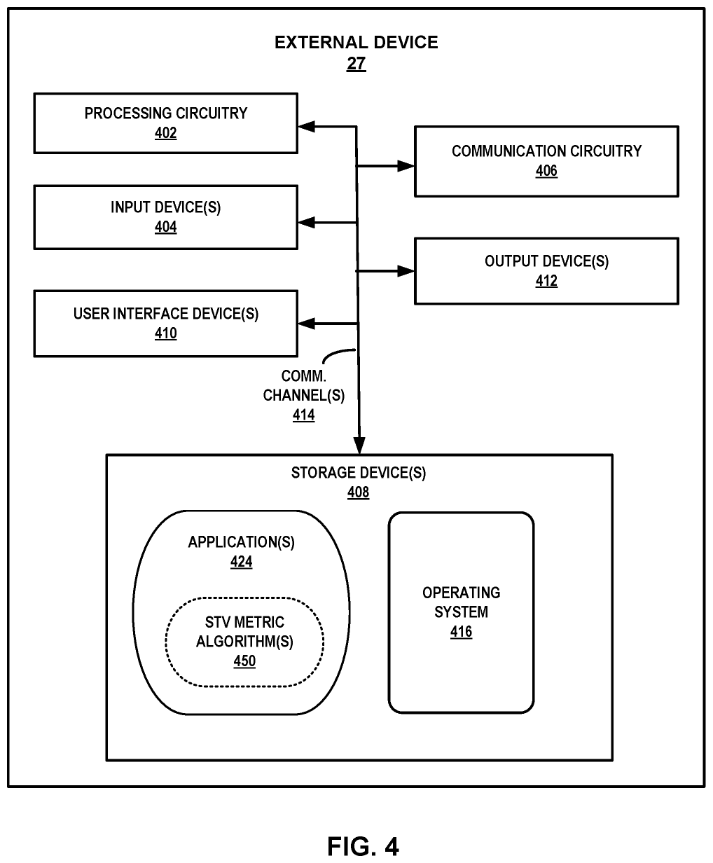

FIG. 4 is a block diagram illustrating an example external device 27 in accordance with one or more techniques of the present disclosure. In some examples, external device 27 takes the form of an external programmer, mobile device, or wearable computing device, such as a mobile phone, a “smart” phone, a laptop, a tablet computer, a personal digital assistant (PDA), a wearable electronic device (e.g., a watch or a patch), an external cardiac monitor, a handheld computing device, computer workstation, server or other networked computing device, etc. In some examples, external device 27 is a CareLink™ monitor available from Medtronic, Inc. An implant could communicate data to such an external programmer, mobile device, or wearable computing device for real-time or for post-processing.

In one example, external device 27 includes processing circuitry 402 for executing applications 424 that STV metric algorithms 450 or any other applications described herein. Although shown in FIG. 4 as a stand-alone external device 27 for purposes of example, external device 27 may be any component or system that includes processing circuitry or other suitable computing environment for executing software instructions and, for example, need not necessarily include one or more elements shown in FIG. 4 (e.g., communication circuitry 406; and in some examples components such as storage device(s) 408 may not be co-located or in the same chassis as other components).

As shown in the example of FIG. 4, external device 27 includes processing circuitry 402, one or more input devices 404, communication circuitry 406, one or more output devices 412, one or more storage devices 408, and user interface (UI) device(s) 410. External device 27, in one example, further includes one or more application(s) 424 such as STV metric algorithms 450, and operating system 416 that are executable by external device 27. Each of components 402, 404, 406, 408, 410, and 412 are coupled (physically, communicatively, and/or operatively) for inter-component communications. In some examples, communication channels 414 may include a system bus, a network connection, an inter-process communication data structure, or any other method for communicating data. As one example, components 402, 404, 406, 408, 410, and 412 may be coupled by one or more communication channels 414.

Processing circuitry 402, in one example, is configured to implement functionality and/or process instructions for execution within external device 27. For example, processing circuitry 402 may be capable of processing instructions stored in storage device 408. Examples of processing circuitry 402 may include, any one or more of a microprocessor, a controller, a digital signal processor (DSP), an application specific integrated circuit (ASIC), a field-programmable gate array (FPGA), or equivalent discrete or integrated logic circuitry.

One or more storage devices 408 may be configured to store information within external device 27 during operation. Storage device 408, in some examples, is described as a computer-readable storage medium. In some examples, storage device 408 is a temporary memory, meaning that a primary purpose of storage device 408 is not long-term storage. Storage device 408, in some examples, is described as a volatile memory, meaning that storage device 408 does not maintain stored contents when the computer is turned off. Examples of volatile memories include random access memories (RAM), dynamic random access memories (DRAM), static random access memories (SRAM), and other forms of volatile memories known in the art. In some examples, storage device 408 is used to store program instructions for execution by processing circuitry 402. Storage device 408, in one example, is used by software or applications 424 running on external device 27 to temporarily store information during program execution.

Storage devices 408, in some examples, also include one or more computer-readable storage media. Storage devices 408 may be configured to store larger amounts of information than volatile memory. Storage devices 408 may further be configured for long-term storage of information. In some examples, storage devices 408 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

External device 27, in some examples, also includes communication circuitry 406. External device 27, in one example, utilizes communication circuitry 406 to communicate with external devices, such as IMD 16 of any one or more of FIGS. 1-3. Communication circuitry 406 may include a network interface card, such as an Ethernet card, an optical transceiver, a radio frequency transceiver, or any other type of device that can send and receive information. Other examples of such network interfaces may include 3G, 4G, 5G, and WiFi radios.

External device 27, in one example, also includes one or more user interface devices 410. User interface devices 410, in some examples, are configured to receive input from a user through tactile, audio, or video feedback. Examples of user interface device(s) 410 include a presence-sensitive display, a mouse, a keyboard, a voice responsive system, video camera, microphone or any other type of device for detecting a command from a user. In some examples, a presence-sensitive display includes a touch-sensitive screen.

One or more output devices 412 may also be included in external device 27. Output device 412, in some examples, is configured to provide output to a user using tactile, audio, or video stimuli. Output device 412, in one example, includes a presence-sensitive display, a sound card, a video graphics adapter card, or any other type of device for converting a signal into an appropriate form understandable to humans or machines. Additional examples of output device 412 include a speaker, a cathode ray tube (CRT) monitor, a liquid crystal display (LCD), or any other type of device that can generate intelligible output to a user.

External device 27 may include operating system 416. Operating system 416, in some examples, controls the operation of components of external device 27. For example, operating system 416, in one example, facilitates the communication of one or more applications 424 and STV metric algorithms 450 with processing circuitry 402, communication circuitry 406, storage device 408, input device 404, user interface devices 410, and output device 412.

Application(s) 424 may also include program instructions and/or data that are executable by external device 27. Example application(s) 424 executable by external device 27 may include STV metric algorithms 450. Other additional applications not shown may alternatively or additionally be included to provide other functionality described herein and are not depicted for the sake of simplicity.

In accordance with one or more techniques of the disclosure, applications 424 include STV metric algorithms 450. In one example, processing circuitry 402 executes STV metric algorithms 450 to determine an STV metric of the patient based on at least one cardiac signal sensed by an IMD. In one example, to control or correct for one or more confounding factors, the at least one cardiac signal may be sensed during a specified time period. As another example, to control or correct for one or more confounding factors, beats having poor T-wave morphology or presence of PVCs may be excluded from the at least one cardiac signal before the STV metric is determined. Processing circuitry 402 of external device 27 may further determine whether the STV metric satisfies one or more therapy delivery thresholds, and, in response to a determination that the STV metric satisfies one or more of the therapy delivery thresholds, transmit an instruction to an associated IMD (such as any of IMDs 16 in FIG. 1, 2, or 3) to deliver therapy configured to suppress tachyarrhythmia to the patient. As another example of controlling or correcting for one or more confounding factors, one or more patient-specific therapy delivery thresholds based on a patient-specific STV baseline value may be determined, processing circuitry 402 of external device 27 may further determine whether the STV metric satisfies one or more of the patient-specific therapy delivery thresholds, and, in response to a determination that the STV metric satisfies one or more of the patient-specific therapy delivery thresholds, transmit an instruction to an associated IMD (such as any of IMDs 16 in FIG. 1, 2, or 3) to deliver therapy configured to suppress tachyarrhythmia to the patient.

In some examples, IMD 16 may perform relatively less complex algorithms to determine an STV metric due to battery and processing power constraints, while external device 27 may not be so limited. For example, external device 27 may be more easily charged, have a larger battery, or have significantly more computing resources. Therefore, external device 27 may apply an algorithm that is more computationally-expensive, algorithmically complex, consumes more power, or analyzes the at least one cardiac signal over a longer period of time than IMD 16 (e.g., minutes or hours for external device 27 versus seconds or minutes for IMD 16). However, it shall be understood that either or both of IMD 16 or external computing device 27 may perform some or all of the STV metric functionality according to the techniques described herein, and that the disclosure is not limited in this respect.

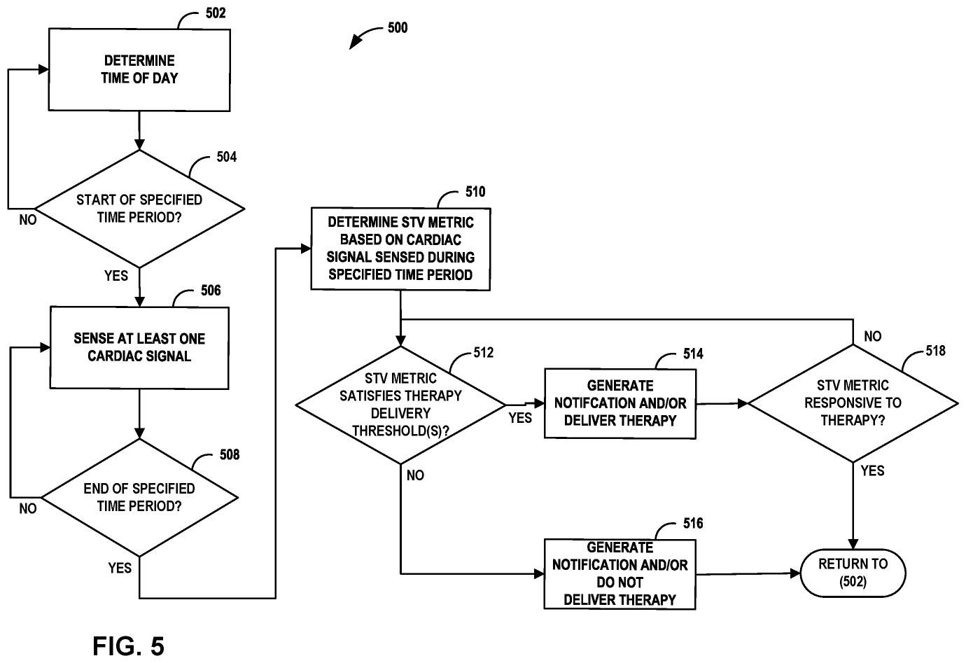

FIG. 5 is a flowchart illustrating an example operation in accordance with one or more techniques of the disclosure. More specifically, FIG. 5 illustrates an example process (500) by which a computing device (such as IMD 16 or external device 27) may determine an STV metric for a patient based on at least one cardiac signal sensed during a specified time period and determine whether to deliver therapy configured to suppress tachyarrhythmia to the patient based on the determined STV metric. For purposes of discussion, example process (500) of FIG. 5 is described herein with respect to IMD 16 of FIGS. 1-3. It shall be understood, however, that in other examples the process (500) of FIG. 5 may be performed by external device 27 of FIGS. 1 and 4, or by a combination of IMD 16 and external device 27.

As depicted in the example of FIG. 5, processing circuitry 58 of IMD 16 determines the time of day (502) and determines whether the time of day corresponds to a start of a specified time period for sensing one or more cardiac signals for use in determining an STV metric (504). If the start of the specified time period has not yet begun (NO branch of 504), processing circuitry 58 continues monitoring the time of day until the start of the specified time period occurs.

At the start of the specified time period (YES branch of 504), IMD 16 begins sensing at least one cardiac signal (506). IMD 16 further determines whether the end of the specified time period has been reached (508). If the end of the specified time period has not been reached (NO branch of 508), IMD 16 continues sensing the at least one cardiac signal (506).

When the end of the specified time period has been reached (YES branch of 508), IMD 16 determines an STV metric based on the at least one cardiac signal sensed during the specified time period (510). IMD 16 determines whether the determined STV metric satisfies one or more therapy delivery thresholds (512). When IMD 16 determines that the determined STV metric satisfies one or more STV therapy delivery thresholds (YES branch of 512), IMD 16 delivers therapy configured to suppress tachyarrhythmia, e.g., overdrive pacing, other cardiac pacing, or neurostimulation, to the patient (514). In addition or alternatively, in some examples, the therapy may include any type of medical therapy, including but not limited to drug therapy, magnetic therapy, and/or any other type of non-electrical therapy, that may be delivered to the patient.

For example, upon determining that the STV metric satisfies one or more therapy delivery thresholds, processing circuitry 58 of IMD 16 generates a corresponding notification and/or controls therapy delivery circuitry 52 to deliver electrical therapy to the patient via one or more of electrodes 40, 42, 44-48 and/or 50. When IMD 16 determines that the determined STV metric does not satisfy one or more therapy delivery thresholds (NO branch of 512), IMD 16 generates a corresponding notification and/or does not deliver electrical therapy to the patient (516). For example, in response to determining that the determined STV metric does not satisfy one or more therapy delivery thresholds (NO branch of 512), processing circuitry 58 does not take action and does not deliver therapy to the patient at the present time (516).

In some examples, after delivery of the therapy (514) IMD 16 may continue to monitor the STV to determine whether the STV metric has been reduced following delivery of the therapy (518). If processing circuitry 52 of IMD 16 determines that the STV metric has been reduced (YES branch of 518), IMD 16 may return to normal monitoring of the STV metric (502). If processing circuitry 52 of IMD 16 determines that the STV metric has not been reduced following delivery of the therapy (NO branch of 518), processing circuitry may continue instructing therapy delivery circuits to deliver the therapy to the patient in an attempt to reduce the STV metric (514).

In some examples, the determination as to whether the STV metric satisfies one or more therapy delivery thresholds (512) may include identifying one or trends in the STV metric over one or more days or weeks and/or one or more comparison(s) of the STV metric to the STV metric(s) determined for previous days. For example, one or more patient-specific therapy delivery thresholds may be determined based on an increase (e.g., fixed value or percentage) over a programmed patient's baseline STV value(s) over the course of one or more days. As another example, one or more patient-specific therapy delivery thresholds may be determined based on a trending increase (e.g., fixed value or percentage) of the patient's STV value(s) over one or more prior days. As another example, one or more patient-specific therapy delivery thresholds may be automatically programmed after an arrhythmic event is detected by the device. In such examples, the device may detect an arrhythmic event which was not predicted ahead of time, and then automatically adjust and re-program the STV threshold for the patient based on the patient's STV and the detected arrhythmic event. In addition, the determining as to whether the STV metric satisfies one or more therapy delivery thresholds may also be based on a patient population to which the patient belongs. For example, the therapy delivery thresholds may be based on different patient populations, such as age, gender, amount of scar tissue, comorbidities, etc.) and the system may determine the patient-specific therapy delivery thresholds based on the patient's membership in one or more patient populations.

In another example, generating a corresponding notification (514, 516) includes generating a corresponding notification regarding the STV metric or an arrhythmic risk level determined based on STV metric to one or more computing devices. The computing device(s) may include, for example, an external computing device such as external device 27 as shown in FIG. 1. As discussed herein, external device 27 may include an external programmer or mobile device, such as a mobile phone, a “smart” phone, a laptop, a tablet computer, a personal digital assistant (PDA), a wearable electronic device, a handheld computing device, computer workstation, server or other networked computing device, etc. A user, such as a patient, physician, technician, surgeon, electro-physiologist, or other clinician, may receive the notification(s) and make informed decisions based on the information contained therein. In such examples, the process may, but need not necessarily, include delivering therapy configured to suppress tachyarrhythmia to the patient (514). The notification may be for example, for periodic monitoring purposes or for notification of desirable and/or undesirable trends in the patient's STV values over a period of time.

In another example, determining whether the STV metric satisfies one or more therapy delivery thresholds (512) may also include, upon determining that the STV metric satisfies one or more therapy delivery thresholds, implementing one or more computationally complex detection algorithm(s) for the periods of time determined to be “high risk”. This may result in more accurate determination of the patient's arrhythmic risk as well as help minimize use of computational resources when an initial therapy delivery threshold is satisfied.

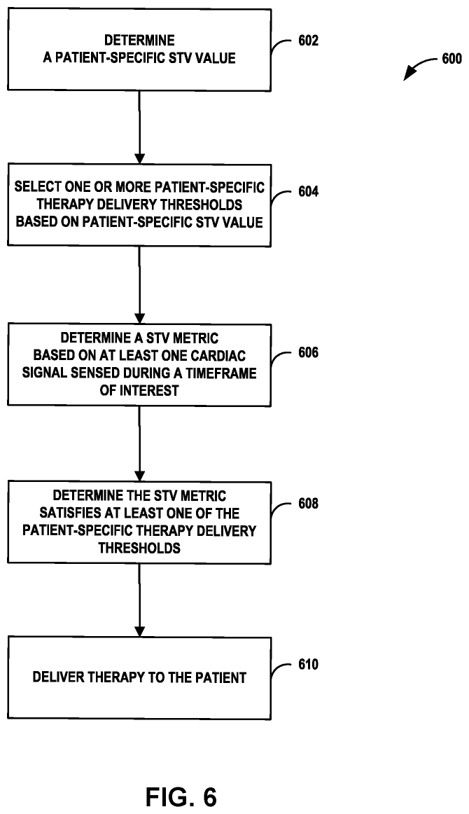

FIG. 6 is a flowchart illustrating an example process (600) by which a computing device (such as IMD 16 or external device 27) may control or correct for one or more confounding factors based on a patient-specific baseline STV value in accordance with one or more techniques of this disclosure. For purposes of discussion, example process (600) of FIG. 5 is described herein with respect to IMD 16 of FIGS. 1-3. It shall be understood, however, that in other examples the process (500) of FIG. 5 may be performed by external device 27 of FIGS. 1 and 4, or by a combination of IMD 16 and external device 27.

Processing circuitry 52 may determine a patient-specific baseline STV value based on at least one cardiac signal sensed during a control timeframe when the patient was not experiencing a tachyarrhythmia event (602). Processing circuitry 52 may select one or more patient-specific therapy delivery thresholds based on the patient-specific baseline STV values (604). For example, one or more patient-specific therapy delivery thresholds may be determined based on an increase (e.g., fixed value or percentage) over a programmed patient's baseline STV value(s). As another example, one or more patient-specific therapy delivery thresholds may be determined based on an increase (e.g., fixed value or percentage) over a trend of the patient's STV value(s) from prior days. As another example, one or more patient-specific therapy delivery thresholds may be automatically programmed after an arrhythmic event is detected by the device. In such examples, the device detected an arrhythmic event which was not predicted ahead of time, and then automatically adjusts and re-program the STV threshold for the patient based on the patient's STV and the detected arrhythmic event.

Processing circuitry 52, determines a STV metric for the patient based on at least one cardiac signal sensed during a timeframe of interest (606). Processing circuitry 52 determines whether the STV metric based on the at least one cardiac signal sensed during a timeframe of interest satisfies one or more of the patient-specific therapy delivery thresholds (608). In response to a determination that the STV metric satisfies one or more of the patient-specific therapy delivery thresholds, processing circuitry 52 may instruct therapy delivery circuitry to deliver electrical therapy to the patient (610).

In another example, determining whether the STV metric satisfies one or more therapy delivery thresholds (608) may further include determining whether the STV metric satisfies one or more thresholds and generating a corresponding notification to an external device. In such examples, the process may, but need not necessarily, include delivering therapy to the patient. For example, one or more threshold(s) may not necessarily be related to the delivery of therapy but may be for periodic monitoring purposes or for notification of an undesirable trend in the patient's STV values over a period of time.

In another example, determining whether the STV metric satisfies one or more therapy delivery thresholds (608) may also include, upon determining that the STV metric satisfies one or more therapy delivery thresholds, implementing a more computationally complex detection algorithm for the periods of time determined to be “high risk”. This may result in more accurate determination of the patient's arrhythmic risk as well as help minimize use of computational resources until an initial therapy delivery threshold is satisfied.

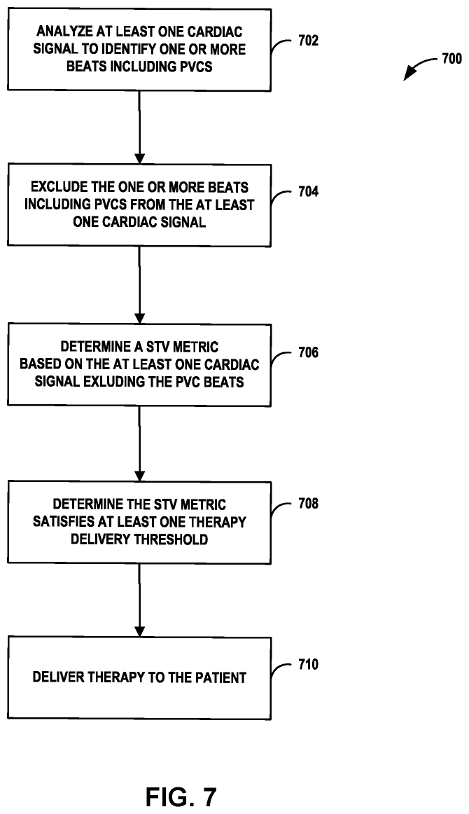

FIG. 7 is a flowchart illustrating an example process (700) by which a computing device (such as IMD 16 or external device 27) may control or correct for a PVC confounding factor in accordance with one or more techniques of this disclosure. Processing circuitry 52 may analyze the at least one cardiac signal to identify one or more beats of the at least one cardiac signal (702) including a PVC occurrence. Processing circuitry 52 may exclude the one or more beats including a PVC occurrence from the at least one cardiac signal in order to avoid STV variation due to PVC disturbances (704). In some examples, this step may further include excluding one or more beats before and after the PVC beats. Processing circuitry 52 may determine a STV metric based on the at least one cardiac signal in which the one or more beats including a PVC occurrence are excluded. Processing circuitry 52 determines whether the STV metric based on the at least one cardiac signal in which the one or more beats including a PVC occurrence are excluded satisfies one or more therapy delivery thresholds (706). In response to a determination that the STV metric satisfies one or more of the therapy delivery thresholds (708), processing circuitry 52 may generate a corresponding notification and/or instruct therapy delivery circuitry to deliver therapy configured to suppress tachyarrhythmia to the patient (710). time.

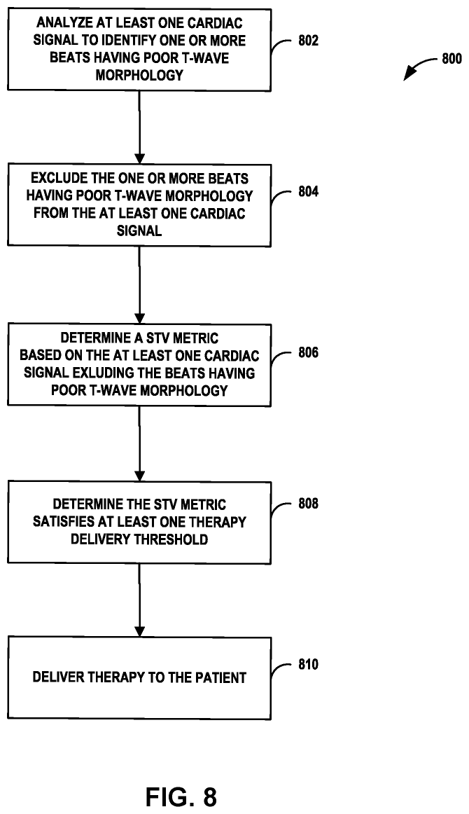

FIG. 8 is a flowchart illustrating an example process (800) by which a computing device (such as IMD 16 or external device 27) may control or correct for a T-wave morphology confounding factor in accordance with one or more techniques of this disclosure. Processing circuitry 52 may analyze the at least one cardiac signal to identify one or more beats of the at least one cardiac signal including a poor T-wave morphology (802). Processing circuitry 52 may exclude the one or more beats including a poor T-wave morphology from the at least one cardiac signal (804). Processing circuitry 52 may determine a STV metric based on the at least one cardiac signal in which the one or more beats including a poor T-wave morphology are excluded. Processing circuitry 52 determines whether the STV metric based on the at least one cardiac signal in which the one or more beats including a poor T-wave morphology are excluded satisfies one or more therapy delivery thresholds (806). In response to a determination that the STV metric satisfies one or more of the therapy delivery thresholds (808), processing circuitry 52 may generate a corresponding notification and/or instruct therapy delivery circuitry to deliver therapy configured to suppress tachyarrhythmia to the patient (810).

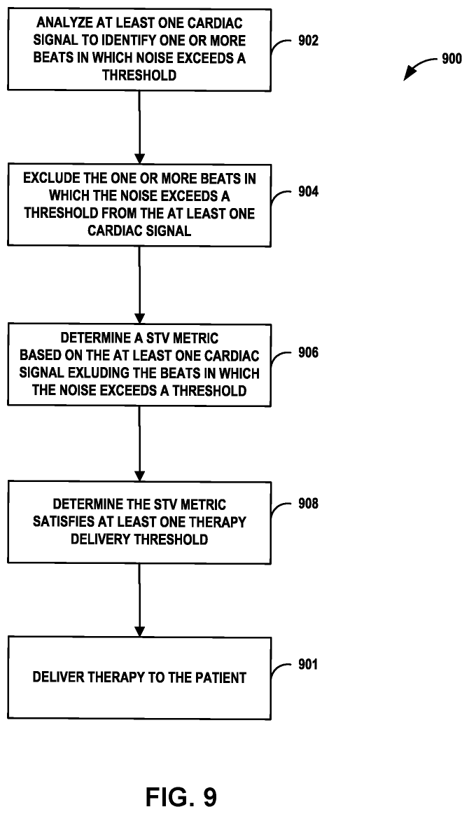

FIG. 9 is a flowchart illustrating an example process (900) by which a computing device (such as IMD 16 or external device 27) may control or correct for noise in the cardiac signal as a confounding factor in accordance with one or more techniques of this disclosure. Processing circuitry 52 may analyze the at least one cardiac signal to identify one or more beats in the at least one cardiac signal in which the noise exceeds a threshold (902). Processing circuitry 52 may exclude the one or more beats in which the noise exceeds a threshold from the at least one cardiac signal (904). Processing circuitry 52 may determine a STV metric based on the at least one cardiac signal in which the one or more beats in which the noise exceeds a threshold are excluded. Processing circuitry 52 determines whether the STV metric based on the at least one cardiac signal in which the one or more beats in which the noise exceeds a threshold are excluded satisfies one or more therapy delivery thresholds (906). In response to a determination that the STV metric satisfies one or more of the therapy delivery thresholds (908), processing circuitry 52 may generate a corresponding notification and/or instruct therapy delivery circuitry to deliver therapy configured to suppress tachyarrhythmia to the patient (910).

Although the processes shown in FIGS. 5-9 are described individually, it shall be understood that any one or more of processes 500, 600, 700, 800 and/or 900 may be implemented together or separately to correct or control for one or more confounding factors. For example, process 500 may be performed alone or in combination with any one or processes 600, 700, 800, and/or 900. Similarly, two, three or all of processes 500, 600, 700, 800 and 900 may be performed to correct or control for several of the confounders listed herein.

By determining the STV metric using any one or more of the confounder control or correction techniques of this disclosure, the effect of the one or more confounding factors on the STV metric determination may be less as compared to STV metrics that are not determined using the confounder control or correction techniques described herein. Thus, according to one or more techniques of the present disclosure, determination of a STV metric using any one or more of the techniques of the present disclosure may result in an STV metric that is more likely to be associated with tachyarrhythmic risk. In turn, the determination as to whether electrical therapy based on the STV metric is more likely to result in a determination that therapy should be delivered when the tachyarrhythmic risk is actually increased. In this way, the techniques of the disclosure may help to prevent false positives in the determination of whether a patient is at risk of tachyarrhythmia or more serious cardiac event, further helping to prevent delivery of unnecessary electrical therapy and avoiding unnecessary physical and psychological distress for the patient.

In some examples, one or more techniques of the disclosure include a system that comprises means to perform any method described herein. In some examples, one or more techniques of the disclosure include a computer-readable medium comprising instructions that cause processing circuitry to perform any method described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Various examples have been described. These and other examples are within the scope of the following claims

What is claimed is:

1. A method comprising:
sensing at least one cardiac signal for a patient during a specified time period;
determining a short-term variability (STV) metric for the patient based on the at least one cardiac signal sensed during the specified time period, wherein determining the STV metric comprises at least one of:
controlling the determined STV metric based on one or more confounding factors, or
correcting the determined STV metric based on the one or more confounding factors,
wherein the one or more confounders comprise T-wave morphology; and
generating a corresponding notification based on the STV metric to one or more computing devices.

2. The method of claim 1, wherein the specified time period is determined based on a time that the patient awakes from sleep.

3. The method of claim 1, wherein sensing at least one cardiac signal includes sensing at least one cardiac signal by an implantable medical device.

4. The method of claim 1, further comprising:
determining that the STV metric satisfies one or more therapy delivery thresholds; and
in response to a determination that the STV metric satisfies one or more therapy delivery thresholds, delivering therapy configured to suppress tachyarrhythmia to the patient.

5. The method of claim 4, wherein the therapy configured to suppress tachyarrhythmia delivered to the patient is electrical therapy.

6. The method of claim 1, further comprising:
determining a patient-specific baseline STV value;
selecting one or more patient-specific therapy delivery thresholds based on the patient-specific baseline STV values;
determining whether the STV metric satisfies one or more of the patient-specific therapy delivery thresholds; and
in response to a determination that the STV metric satisfies one or more of the patient-specific therapy delivery thresholds, delivering the therapy configured to suppress tachyarrhythmia to the patient.

7. The method of claim 1, further comprising:
identifying one or more beats in the at least one cardiac signal including premature ventricular contractions (PVCs);
excluding the one or more beats in the at least one cardiac signal including PVCs from the at least one cardiac signal; and
determining a STV metric based on the at least one cardiac signal from which beats including a PVC are excluded.

8. The method of claim 1, further comprising:
determining a T-wave morphology for each beat of the at least one cardiac signal;
excluding one or more beats in the cardiac signal having poor T-wave morphology; and
determining a STV metric based on the at least one cardiac signal in which beats corresponding to poor T-wave morphology are excluded.

9. The method of claim 1, further comprising:
identifying one or more beats in the at least one cardiac signal in which noise exceeds a threshold;
excluding the one or more beats in the at least one cardiac signal in which the noise exceeds a threshold from the at least one cardiac signal; and
determining a STV metric based on the at least one cardiac signal from which beats in which the noise exceeds a threshold are excluded.

10. The method of claim 1, wherein the one or more therapy delivery thresholds are patient-specific therapy delivery thresholds.

11. The method of claim 1, further comprising:
controlling for one or more confounding factors that may influence a determination as to whether therapy should be delivered to the patient.

12. The method of claim 1, wherein the confounding factors include one or more of noise in the cardiac signal, a circadian variation, or a medication effect.

13. The method of claim 1, wherein the at least one cardiac signal comprises a cardiac electrogram signal.

14. A medical device comprising:
sensing circuitry configured to sense at least one cardiac signal of a patient during a specified time period, the specified time period selected to control at least one of noise in the at least one cardiac signal, a circadian variation in the cardiac signal, or a medication effect on the cardiac signal; and
processing circuitry configured to:
determine a short-term variability (STV) metric for the patient based on the at least one cardiac signal sensed during the specified time period;
control the determined STV metric based on one or more confounding factors, or
correct the determined STV metric based on the one or more confounding factors,
wherein the one or more confounding factors comprise T-wave morphology; and
generate a corresponding notification based on the STV metric to one or more computing devices.

15. The medical device of claim 14, wherein the specified time period is determined based on a time that the patient awakes from sleep.

16. The medical of claim 14, wherein the medical device is an implantable medical device.

17. The medical device of claim 14, wherein the processing circuitry is further configured to:
determine that the STV metric satisfies one or more therapy delivery thresholds; and
in response to a determination that the STV metric satisfies one or more therapy delivery thresholds, deliver therapy configured to suppress tachyarrhythmia to the patient.

18. The medical device of claim 17, wherein the therapy configured to suppress tachyarrhythmia delivered to the patient is electrical therapy.

19. The medical device of claim 14, wherein the processing circuitry is further configured to:
determine a patient-specific baseline STV value;
select one or more patient-specific therapy delivery thresholds based on the patient-specific baseline STV values;
determine the STV metric for the patient based on at least one cardiac signal sensed during a timeframe of interest;
determine whether the STV metric satisfies one or more of the patient-specific therapy delivery thresholds; and
in response to a determination that the STV metric satisfies one or more of the patient-specific therapy delivery thresholds, control delivery of the therapy configured to suppress tachyarrhythmia to the patient.

20. The medical device of claim 14, wherein the processing circuitry is further configured to:
identify one or more beats in the at least one cardiac signal including premature ventricular contractions (PVCs);
exclude the one or more beats in the at least one cardiac signal including PVCs from the at least one cardiac signal; and
determine the STV metric based on the at least one cardiac signal from which beats including a PVC are excluded.

21. The medical device of claim 14, wherein the processing circuitry is further configured to:
determine a T-wave morphology for each beat of the at least one cardiac signal;
exclude one or more beats in the cardiac signal having poor T-wave morphology; and
determine the STV metric based on the at least one cardiac signal in which beats corresponding to poor T-wave morphology are excluded.

22. The medical device of claim 14, wherein the processing circuitry is further configured to:
identify one or more beats in the at least one cardiac signal in which noise exceeds a threshold;
exclude the one or more beats in the at least one cardiac signal in which the noise exceeds a threshold from the at least one cardiac signal; and
determine a STV metric based on the at least one cardiac signal from which beats in which the noise exceeds a threshold are excluded.

23. The medical device of claim 14, wherein the one or more therapy delivery thresholds are patient-specific therapy delivery thresholds.

24. The medical device of claim 14, wherein the processing circuitry is further configured to:
control for one or more confounding factors that may influence a determination as to whether therapy should be delivered to the patient.

25. The medical device of claim 14, wherein the confounding factors include one or more of noise in the cardiac signal, a circadian variation, or a medication effect.

26. The medical device of claim 14, wherein the at least one cardiac signal comprises a cardiac electrogram signal.

27. A computer-readable medium comprising instructions that, when executed by one or more processors, cause the one or more processors to:
sense at least one cardiac signal for a patient during a specified time period;
determine a short-term variability (STV) metric for the patient based on the at least one cardiac signal sensed during the specified time period;
control the determined STV metric based on one or more confounding factors, or correct the determined STV metric based on the one or more confounding factors, wherein the one or more confounders comprise T-wave morphology; and
generate a corresponding notification including the STV metric to one or more computing devices.

28. The computer-readable medium of claim 27, further comprising instructions that, when executed by one or more processors, cause the one or more processors to:
determine that the STV metric satisfies one or more therapy delivery thresholds; and
in response to a determination that the STV metric satisfies one or more therapy delivery thresholds, deliver therapy configured to suppress tachyarrhythmia to the patient.
